(19) United States
(12) Patent Application Publication
(10) Pub. No.: US 2007/0276287 A1
(43) Pub. Date: Nov. 29, 2007

(54) Catheter

(75) Inventors: Brenda L. Donaldson, Harrison Township, MI (US); Sachin Vadodaria, Fox Point, WI (US)

Correspondence Address: GE MEDICAL SYSTEM;C/O FOLEY & LARDNER LLP, 777 EAST WISCONSIN AVENUE, MILWAUKEE, WI, 53202-5306, US

(73) Assignee: General Electric Company

(21) Appl. No.: 11/433,596

(22) Filed: May 12, 2006

(51) Int. Cl. A61B 10/00

(52) U.S. Cl. 600/564

(57) ABSTRACT

A catheter includes a shaft having a proximal end and a distal end. The catheter further includes a control handle coupled to the proximal end of the shaft. The catheter further includes a catheter tip coupled to the distal end of the shaft, wherein the catheter tip is incapable of cutting. The catheter further includes a conductor extending through the shaft and coupled to the handle and catheter tip. The outside diameter of the shaft is less than the outside diameter of the catheter tip.

### FIELD OF THE INVENTION

The present invention relates generally to the field of catheters and specifically to a catheter with a distal end larger than the shaft.

### BACKGROUND OF THE INVENTION

Catheters may be used for such procedures as to guide ablation procedures and to monitor ablations. In the future catheters may be used to perform other functions including tissue characterization, characterization of lesion formation, ARFI (Acoustic Radiation Force Impulse), evaluation of tissue synchronization among others. The quality of medical imaging, for example, ultrasound images, may be in direct proportion to the quantity of arrays; the greater the number of arrays the better the image quality may be. The larger the area where arrays can be placed, the better the image quality, depth and field of view may be. Catheters may be introduced into the body via a sheath that may be placed in the femoral vein. The distal end of the catheter (when used for atrial fibrillation ablations and during ablation monitoring) may remain in the right atrium with the arrays aimed at the area of interest in the left atrium. However, it may be difficult to view a section of the left atrium with a large enough field of view to encompass each pulmonary vein with enough depth to be able to see the distal wall of the left atrium when the atrium is enlarged.

The vein or artery that the imaging catheter is inserted through, such as the femoral vein or artery, may also be used to advance the ablation catheter, and other diagnostic/interventional and/or mapping catheters as the physician deems necessary. In smaller patients the femoral vein may be filled with so many catheters that venous return may be impeded or catheter manipulation for proper placement may be compromised. Similarly in smaller patients or those with diseased arterial systems multiple catheters placed in the artery may compromise the blood supply to the distal extremity. Imaging catheters may be placed in femoral arteries and used to view the lumen of arteries that are undergoing intervention. Interventional cardiologists may use guiding catheters which are placed through the same arterial system as the imaging catheter. Guiding catheters may be designed to fit into the aorta in a specific manner. For example, a catheter may be designed to be located at the opening of the left coronary with the opposite wall of the aorta acting as a support for correct catheter placement. The smaller the shaft of the imaging catheter, the less distortion in the physicians normal placement of the guiding catheter there may be.

Thus there is a need for a catheter capable of viewing a section of the left atrium large enough to encompass each pulmonary vein with enough depth to be able to see to the distal wall of the left atrium when the atrium is enlarged. There is also a need for a catheter design that allows for the placement of multiple catheters within a vein or artery without impeding blood flow or damaging or stretching the vein or artery. There is also a need for a catheter design with a catheter tip of a larger diameter than the catheter shaft that does not impact the direction of blood flow.

### SUMMARY OF THE INVENTION

One embodiment of the invention relates to a catheter. The catheter includes a shaft having a proximal end and a distal end. The catheter also includes a control handle coupled to the proximal end of the shaft. The catheter also includes a catheter tip coupled to the distal end of the shaft, wherein the catheter tip is incapable of cutting. The catheter also includes a conductor extending through the shaft and coupled to the handle and catheter tip. The outside diameter of the shaft is less than the outside diameter of the catheter tip and includes a transition portion between the shaft and the catheter tip not greater than 5 millimeters, with the catheter tip and shaft configured as a unitary body.

Another embodiment of the invention relates to a system. The system includes a patient bed configured to support a patient in a patient care facility. The system also includes a medical imaging device configured to obtain internal medical images of the patient. The system also includes a catheter configured to be inserted into the patient to cooperate with the medical imaging device to obtain the medical images. The catheter includes a shaft having a proximal end and a distal end. The catheter also includes a control handle coupled to the proximal end of the shaft. The catheter also includes a catheter tip coupled to the distal end of the shaft, wherein the catheter tip is incapable of cutting. The catheter also includes a conductor extending through the shaft and coupled to the handle and catheter tip. The outside diameter of the shaft is less than the outside diameter of the catheter tip.

Another embodiment of the invention relates to a catheter. The catheter includes a shaft having a proximal end and a distal end. The catheter further includes a control handle coupled to the proximal end of the shaft. The catheter further includes a catheter tip coupled to the distal end of the shaft, wherein the catheter tip is incapable of cutting. The catheter further includes a conductor extending through the shaft and coupled to the handle and catheter tip. The outside diameter of the shaft is less than the outside diameter of the catheter tip.

### BRIEF DESCRIPTION OF THE DRAWINGS

FIG. 1 is a perspective view of a system that includes a catheter for use on a patient according to one exemplary embodiment.

FIG. 2 is aside perspective view of the catheter of FIG. 1 according to one exemplary embodiment.

### DETAILED DESCRIPTION OF THE PREFERRED EMBODIMENTS

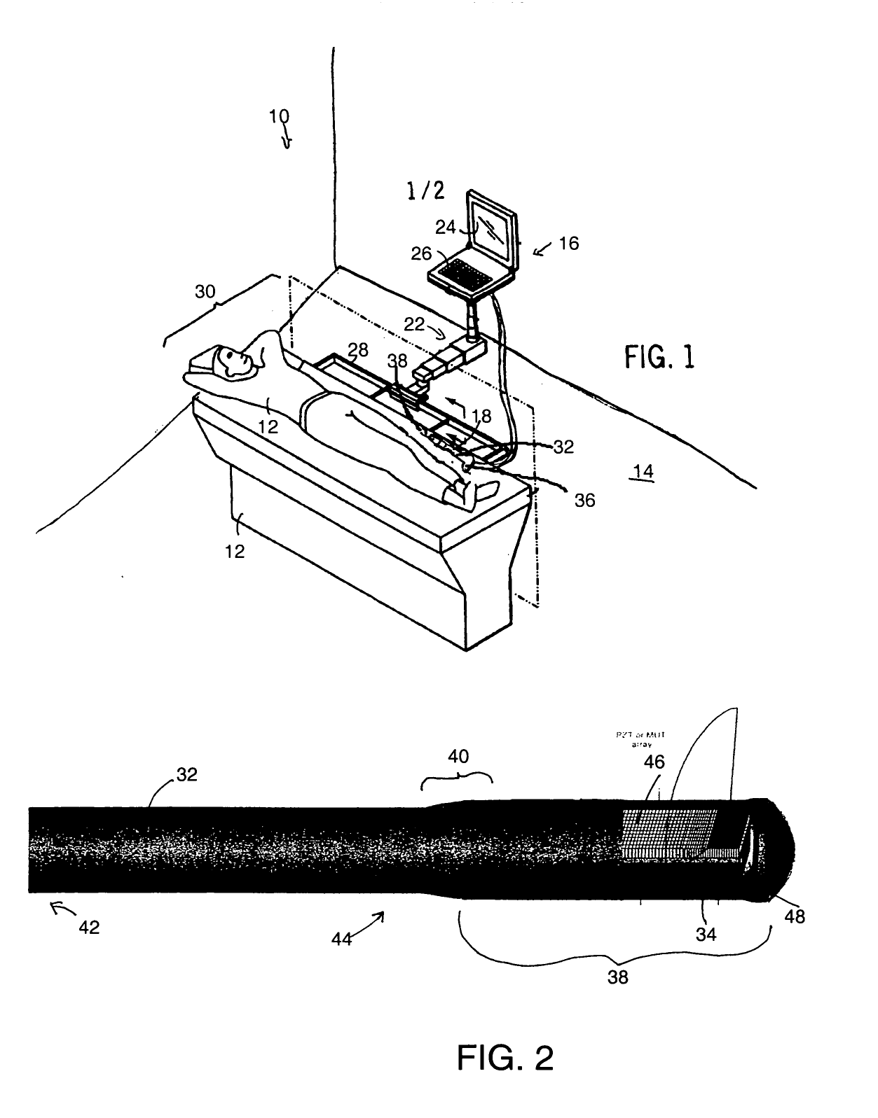

Referring to FIG. 1, a system 10 includes a patient bed 12, a patient care facility 14, a medical imaging device 16, and a catheter 18. Patient bed 12 is configured to support a patient 20 in patient care facility 14 so that medical imaging device 16 may obtain internal medical images of patient 20 using catheter 18. In various exemplary embodiments, patient bed 12 may be any suitable bed of past, present, or future design that is capable of supporting a patient. In one exemplary embodiments, patient 20 may be a human. In other exemplary embodiments, patient 20 may be another anatomical structure such as a dog, cat, horse, or primate. In another exemplary embodiment, patient care facility 14 may be a hospital or hospital room, while in still other exemplary embodiments, facility 14 may be any facility suitable for performing medical imaging on patient 20.

Medical imaging device 16 may include a mounting structure 22 and may cooperate with imaging sensor or catheter 18 to obtain medical images from patient 20. Imaging device 16 may be of a laptop-style configuration with a flip-up screen 24 that displays imaging data or options and a horizontal work area 26. In various exemplary embodiments imaging device 16 may be any imaging device of past, present, or future design such as an ultrasound imaging device, x-ray imaging device, or thermal imaging device. In one example embodiment imaging device 16 may be configured to process the data retrieved using an imaging processor.

Mounting structure 22 is configured to support imaging device 16 and is coupled to bed 12 via a rail 28 in an area external to a sterile zone or field 30 encompassing bed 12. In another exemplary embodiment, mounting structure 22 may be coupled to bed 12 via means other than a rail. Mounting structure 22 is capable of positioning imaging device 16 in a variety of locations as desired by a clinician for various reasons such as for best viewing or so as to not be obstructing any procedure performed on patient 20. In other exemplary embodiments, mounting structure may be a cart that is not directly coupled to bed 12.

Catheter 18 may be configured to be inserted into patient 20 to cooperate with medical imaging device 16 and obtain medical imaging data. Catheter 18 may include a shaft 32, a conductor 34, a control handle 36, a catheter tip 38, and a transition portion 40. Shaft 32 is configured to couple the catheter tip 38 to the control handle 36 and includes a proximal end 42 and a distal end 44. Proximal end 42 is coupled to the catheter tip 38 while distal end 44 is coupled to the control handle 36. In one exemplary embodiment, catheter tip 38 or control handle 36 may not be removable from shaft 32, while in another exemplary embodiment, tip 38 and handle 36 may be removable from shaft 32. In various exemplary embodiments shaft 32 may be composed of any flexible material such as plastic, rubber, a suitable metal, or any combination thereof. Shaft 32 encloses conductor 34.

Conductor 34 extends through shaft 32 and is coupled to handle 36 and tip 38. Conductor 34 may be any type of wire or bus that provides or receives electrical signals to and from catheter tip 38 and handle 36. In one exemplary embodiment, conductor 34 may be configured as a power conductor. In another exemplary embodiment, conductor 34 may be configured as a data conductor. In still other exemplary embodiments, conductor 34 may be a steering wire for guiding catheter tip 38 or a safety wire for retrieval of catheter tip 38. In various exemplary embodiments, shaft 32 may include one or more of any one or any combination of the above conductor types.

Control handle 36 is configured to allow gripping and controlling of catheter 18 by a user. Control handle 36 may be manipulated by a user to guide catheter tip 38 to a desired location or to retrieve data from within patient 20, such as imaging data. In one exemplary embodiment, handle 36 may include a joystick or other control device to allow for catheter tip guidance or data retrieval. In other exemplary embodiments, handle 36 may be of any desired ergonomic or non-ergonomic shape. In still other exemplary embodiments handle 36 may be composed of any material suitable for gripping such as plastic, rubber, metal, wood, or any combination thereof.

Referring also to FIG. 2, catheter tip 38 is an overmolded portion of catheter 18 coupled to the distal end of shaft 32 that is configured to perform the actual data retrieval from patient 20. In general, catheter tip 38 may be incapable of cutting, for example with a blade. Catheter tip 38 may have a diameter larger than the diameter of shaft 32. For purposes of this disclosure, the term “diameter” means the length of a straight line through the center of an object. In one exemplary embodiment where catheter tip 38 and shaft 32 may be cylindrical, the diameter is a perpendicular line straight through any point on the longitudinal center line of the two cylinders. In another exemplary embodiment where tip 38 and shaft 32 may be prismatic, the diameter is a perpendicular straight line through any point of the prism so long as a line of the same slope and offset is drawn through both tip 38 and shaft 32. In one exemplary embodiment, the outside diameter of shaft 32 may be less than the outside diameter of catheter tip 38 within a range of one to three millimeters. In another exemplary embodiment where tip 38 and shaft 32 are cylindrical, the outside diameter of shaft 32 may be at least two French less than the outside diameter of tip 38.

Catheter tip 38 includes a sensor 46 and a window 48. Sensor 46 performs the data retrieval by sensing various conditions, factors, or attributes. Sensor 46 may be any type of sensor, including but not limited to a transducer or a probe, that is able to transmit and receive data. In one exemplary embodiment sensor 46 may be an ultrasound array configured to emit and sense sound waves in a manner to facilitate ultrasonic imaging. In another exemplary embodiment, sensor 46 may detect infrared light waves to facilitate thermal imaging. In still other exemplary embodiments, sensor 46 may emit and sense radiation to facilitate X-ray imaging or may sense electric activity.

Window 48 may be a transparent portion of catheter tip 38 that allows light waves of one or more wavelengths—such as visible, infrared, and x-ray light waves, for example—and/or sound waves to pass through to and from exposed sensor 46. For purposes of this application transparent window means that window 48 is not opaque to the type of energy, e.g., sound, or light, being transmitted and received through window 48. In one exemplary embodiment window 48 may be configured to extend around a 180 degree portion of catheter tip 38. In other exemplary embodiments, window 48 may extend around greater or less than a 180 degree portion of catheter tip 38.

Transition portion 40 is intended to provide a relatively smooth slope or transition between tip 38 and shaft 32.so that an impact on the direction of blood flow, for example turbulence or cavitation, is reduced. In various exemplary embodiments transition portion 40 and tip 38 may be of any material such as plastic, rubber, metal, or any combination thereof.

Although specific shapes of each element have been set forth in the drawings, each element may be of any other shape that facilitates the function to be performed by that element. For example, catheter tip 38 is shown to have a cylindrical shape, however, in other embodiments tip 38 may be of a prismatic shape, for example, a rectangular cross-section.

Although system 10 is illustrated as including multiple features utilized in conjunction with one another, system 10 may alternatively utilize less than all of the noted mechanisms or features. For example, in other exemplary embodiments, bed rail 28 may be omitted with imaging device 16 mounted to bed 12 or other structure via another means.

For purposes of this disclosure, the term “coupled” means the joining of two components (electrical or mechanical) directly or indirectly to one another. Such joining may be stationary in nature or movable in nature. Such joining may be achieved with the two components (electrical or mechanical) and any additional intermediate members being integrally defined as a single unitary body with one another or with the two components or the two components and any additional member being attached to one another. Such joining may be permanent in nature or alternatively may be removable or releasable in nature

The present disclosure has been described with reference to example embodiments, however workers skilled in the art will recognize that changes may be made in form and detail without departing from the spirit and scope of the invention. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example is manifestly intended to be as broad as possible. For example, unless specifically otherwise noted a single particular element may also encompass a plurality of such particular elements.

It is also important to note that the construction and arrangement of the elements of the system as shown in the preferred and other exemplary embodiments is illustrative only. Although only a certain number of embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the assemblies may be reversed or otherwise varied, the length or width of the structures and/or members or connectors or other elements of the system may be varied, the nature or number of adjustment or attachment positions provided between the elements may be varied. It should be noted that the elements and/or assemblies of the system may be constructed from any of a wide variety of materials that provide sufficient strength or durability. Accordingly, all such modifications are intended to be included within the scope of the present disclosure. Other substitutions, modifications, changes and omissions may be made in the design, operating conditions and arrangement of the preferred and other exemplary embodiments without departing from the spirit of the present subject matter.

What is claimed is:

1. A catheter comprising:
a shaft having a proximal end and a distal end;
a control handle coupled to the proximal end of the shaft;
a catheter tip coupled to the distal end of the shaft, wherein the catheter tip is incapable of cutting; and
a conductor extending through the shaft and coupled to the handle and catheter tip, wherein the outside diameter of the shaft is less than the outside diameter of the catheter tip and including a transition portion between the shaft and the catheter tip not greater than five millimeters, with the catheter tip and shaft configured as a unitary body.

2. The catheter of claim 1, wherein the outside diameter of the shaft is less than the outside diameter of the catheter tip within a range of one to three millimeters.

3. The catheter of claim 2, wherein the outside diameter of the shaft is at least two French less than the outside diameter of the catheter tip.

4. The catheter of claim 1, including an ultrasound array encapsulated in the catheter tip and coupled to the conductor.

5. The catheter of claim 1, wherein the catheter tip is not removable from the shaft.

6. A system comprising:
a patient bed configured to support a patient in a patient care facility;
a medical imaging device configured to obtain internal medical images of the patient; and
a catheter configured to be inserted into the patient to cooperate with the medical imaging device to obtain the medical images, the catheter comprising:
a shaft having a proximal end and a distal end;
a control handle coupled to the proximal end of the shaft;
a catheter tip coupled to the distal end of the shaft, wherein the catheter tip is incapable of cutting; and
a conductor extending through the shaft and coupled to the handle and catheter tip, wherein the outside diameter of the shaft is less than the outside diameter of the catheter tip.

7. The system of claim 6, wherein the outside diameter of the shaft is less than the outside diameter of the catheter tip within a range of one to three millimeters.

8. The system of claim 7, wherein the outside diameter of the shaft is at least two French less than the outside diameter of the catheter tip.

9. The system of claim 7, including a transition portion between the shaft and the catheter tip not greater than 5 millimeters.

10. The system of claim 6, including an ultrasound array encapsulated in the catheter tip and coupled to the conductor.

11. The system of claim 6, wherein the catheter tip and shaft are a unitary body.

12. The system of claim 6, wherein the catheter tip is not removable from the shaft.

13. A catheter comprising:
a shaft having a proximal end and a distal end;
a control handle coupled to the proximal end of the shaft;
a catheter tip coupled to the distal end of the shaft, wherein the catheter tip is incapable of cutting; and
a conductor extending through the shaft and coupled to the handle and catheter tip, wherein the outside diameter of the shaft is less than the outside diameter of the catheter tip.

14. The catheter of claim 13, wherein the outside diameter of the shaft is less than the outside diameter of the catheter tip within a range of one to three millimeters.

15. The catheter of claim 14, wherein the outside diameter of the shaft is at least two French less than the outside diameter of the catheter tip.

16. The catheter of claim 14, including a transition portion between the shaft and the catheter tip not greater than 5 millimeters.

17. The catheter of claim 13, including an ultrasound array encapsulated in the catheter tip and coupled to the conductor.

18. The catheter of claim 13, wherein the catheter tip and shaft are a unitary body.

19. The catheter of claim 13, wherein the catheter tip is not removable from the shaft.
